(19) United States
(12) Patent Application Publication
(10) Pub. No.: US 2022/0367055 A1
(43) Pub. Date: Nov. 17, 2022

(54) NEURODEGENERATION AND CHIROGRAPHIC PERFORMANCE MONITORING

(71) Applicant: Huma Therapeutics Ltd., London (GB)

(72) Inventors: Tamir Strauss, London (GB); Emily Binning, London (GB); Danoosh Vahdat, London (GB); Robert Huddlestone, London (GB)

(73) Assignee: Huma Therapeutics Ltd., London (GB)

(21) Appl. No.: 17/824,426

(22) Filed: May 25, 2022

Related U.S. Application Data
(63) Continuation-in-part of application No. PCT/GB2020/052996, filed on Nov. 25, 2020.
Application No. PCT/GB2020/052996 is a continuation-in-part of application No. PCT/GB2020/052995, filed on Nov. 25, 2020.

(30) Foreign Application Priority Data
Nov. 25, 2019 (GB) 1917145.3
Nov. 25, 2019 (GB) 1917146.1

(51) Int. Cl. G16H 50/20; A61B 5/11

(52) U.S. Cl. CPC G16H 50/20; A61B 5/1124

(57) ABSTRACT

A computer system for monitoring a change in a neurodegenerative disease in a user, comprising a writing apparatus comprising a sensor for recording data values indicating the chirographic performance of the user; a storage medium configured to store predetermined relationships associating the change in chirographic performance of the user over time with an indication of the change in the neurodegenerative disease of a user; and a processor connected to the sensor. A computer system for monitoring a change in a neurodegenerative disease in a user, comprising a display for presenting visual content to the user; a sensor for recording data indicating chirographic performance of the user, and intellectual performance of the user; a storage medium configured to store predetermined relationships associating the chirographic and intellectual performance with an indication of the change in the neurodegenerative disease; and a processor connected to both the display and the sensor.

### FIELD OF THE INVENTION

This invention relates to monitoring neurodegeneration, and in particular to a computer system for monitoring a change in the neurodegeneration in a patient.

### BACKGROUND OF THE INVENTION

Neurodegeneration is defined as the progressive loss of structure or function of neurons located in the brain and spinal cord. Neurons are not reproducible or replaceable, and so the damage of these cells results in the progressive degeneration of the ability of an individual suffering with the condition to perform tasks requiring movement and/or mental functioning. Neurodegeneration is an incurable and irreversible condition, and is caused by a number of neurodegenerative diseases, such as Alzheimer's disease, Parkinson's disease and Huntington's disease.

The diagnosis and subsequent monitoring of a neurodegenerative disease in an individual at present is typically conducted during an appointment between the individual and a medical professional and comprises a number of cognitive assessments to assess the mental abilities of the individual. These mental abilities include short and long-term memory, concentration and attention span, language and communication skills, awareness of time and place, and visuospatial abilities. The diagnosis of neurodegenerative diseases in an individual may take multiple appointments and many months of testing. Following diagnosis, a course of treatment or therapy can be prescribed to the individual, and they can organise their lifestyle appropriately to adapt to their conditions.

A significant problem with the present assessments of neurodegenerative diseases that are currently in use is that they are highly subjective. That is, the outputs of these assessments are subject to the opinion of the medical professional as to how the patient has responded to the tasks or questions that they are asked to complete. As such, these tests may be inaccurate as to how advanced the neurodegeneration of the individual is. In addition to this, the involvement of a medical professional in the monitoring of a patient with neurodegeneration results in a significant time and cost inefficiency. That is, it may take a considerable amount of time for a patient to secure an appointment with a medical professional. Furthermore, the appointment may incur a cost to the patient. These considerations add to the complexity associated with the diagnosis and treatment of neurodegenerative diseases.

There is a need for an improved method of monitoring neurodegeneration in a patient.

### SUMMARY OF THE INVENTION

According to a first aspect of the present invention there is provided a computer system for monitoring a change in a neurodegenerative disease in a user, the computer system comprising: a writing apparatus comprising a sensor for recording data values indicating the chirographic performance of the user; and a storage medium configured to store a plurality of predetermined relationships associating the change in chirographic performance of the user over time with an indication of the change in the neurodegenerative disease of a user; a processor connected to the sensor, wherein the processor is configured to: collect data values from the sensor at a plurality of distinct points in time indicating the chirographic performance of the user at each distinct point in time; use a plurality of data values collected from the sensor at each distinct point in time to determine a change in the chirographic performance of the user over time; and compare the change in the chirographic performance of the user over time with the plurality of predetermined relationships that are stored in the storage medium to establish the change in the neurodegenerative disease in the user.

The neurodegenerative disease may be Alzheimer's disease.

The neurodegenerative disease may be Parkinson's disease.

The neurodegenerative disease may be Huntington's disease.

The system may further comprise a display for presenting visual content to a user.

The visual content may comprise instructions for a user to perform one or more writing assessment tasks.

The one or more writing assessment tasks may include assessing the speed with which the user is capable of forming sentences or words.

The data values indicating the chirographic performance of the user may comprise values representing one or more of the following parameters: the roundness or sharpness of letters, the spacing between letters, the slope of letters, repetition or irregularity of elements, the pressure applied during writing, the average size of letters, the thickness of letters, and the speed of formation of letters.

Establishing the change in the neurodegenerative disease may comprise establishing a discrete absolute score of neurodegeneration.

Establishing the change in the neurodegenerative disease may comprise establishing a patient-specific relative score of neurodegeneration.

The change in the neurodegenerative disease may be a progression of that neurodegenerative disease.

The change in the neurodegenerative disease may be a regression of that neurodegenerative disease.

The processor and the storage medium may be comprised within a device.

The writing apparatus may be a digital pen.

The sensor may be configured to start collecting data when an application that is configured to be run on the device is open.

The visual content may be preprogramed into an application to be run on the device.

### DETAILED DESCRIPTION

The present invention will now be described by way of example with reference to the accompanying drawings.

In the drawings:

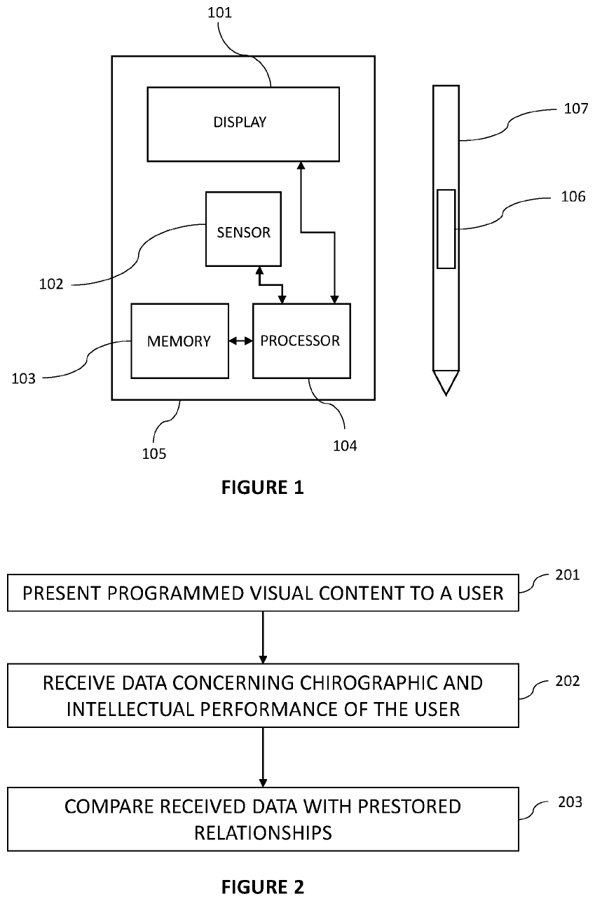

FIG. 1 illustrates an arrangement of the neurodegeneration monitoring system.

FIG. 2 illustrates an exemplary method for monitoring neurodegeneration using the described system.

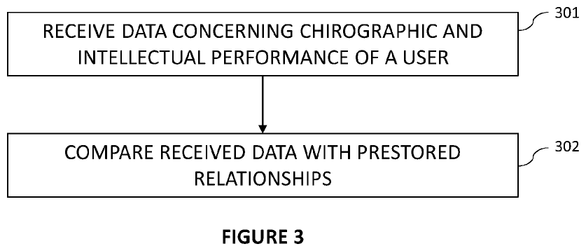

FIG. 3 illustrates an alternative exemplary method for monitoring neurodegeneration using the described system.

This application concerns a system for the remote monitoring of neurodegeneration in a patient, without the need for the external intervention of a medical professional. This system is illustrated in FIG. 1, and comprises a display 101, an image sensor 102, a storage medium such as memory 103 (which may be implemented on one or more integrated circuits and/or on a hard drive) and a processor 104 (which may comprise one or more data processing integrated circuits and/or one or more graphics processing integrated circuits). In one example of the invention, these components may be comprised within one device 105.

The display 101 of the system may also be a user interface, such as the screen of a smartphone device. Alternatively, the display 101 may fulfil a visual function only, and may form part of a personal or laptop computer. The display 101 is electrically connected to the processor 104 to enable the exchange of data in both directions between the display 101 and the processor 104 and is controlled by the processor 104 to present one or more items of visual content to a user. The visual content may comprise instructions for a user to perform a certain task. For example, the user may be instructed to perform one or more writing assessment tasks. The writing assessment tasks may comprise forming sentences or drawing a repeated letter, for example. The writing assessment may assess the grammar that the user is capable of using, or alternatively may assess the vocabulary of the user. Alternatively, the writing assessment may assess the speed with which the user is capable of forming sentences or words. One or more writing assessment variations may be implemented.

The sensor 102 is configured to record data indicating the reaction of a user to visual content that is presented on the display 101. More specifically, the sensor 102 may record the response of a user to the one or more writing assessment tasks. In one example of the invention, the sensor may be a touch sensor, which is a transducer that is sensitive to pressure or human touch. Alternatively, the sensor may be any other sensor that can record the writing of the user. The term writing may be used to describe the handwriting, or alternatively the typing performance of the user. The sensor 102 may be located under the keys of a keypad to record the force applied to the keys of the keypad when the user types. The sensor 102 alternatively may be integrated into the display 101 to form a touchscreen. In this example the sensor 102 and the display 101 are located within the same device 105. In this example, the sensor 102 may be capable of detecting the difference between a hand or a pen contacting the display 101. The sensor 102 may also be capable of sensing the variations between individual text characters. The sensor 102 is electrically connected to the processor 104 to enable the exchange of data in both directions between the sensor 102 and the processor 104.

In an alternative example, the computer system may comprise an additional sensor 106 which may be located in a second device, or writing apparatus 107, that is configured to interact with the first device 105. For example, the second sensor 106 may be located in an electronic writing device for writing on the display 101 of the device 105. The electronic writing device may be a stylus, or alternatively may be digital smart pen. The sensor 102 or 106 is configured to record one or more parameters indicating the chirographic performance of the user. These parameters may include one or more of the following:
(i) the roundness or sharpness of letters,
(ii) the spacing between letters,
(iii) the slope of letters,
(iv) repetition or irregularity of elements,
(v) the pressure applied during writing,
(vi) the average size of letters,
(vii) the thickness of letters.
(viii) the speed of formation of letters

In addition to the above factors, the sensor 102 or 106 may be configured to record one or more parameters indicating the intellectual performance of the user. The intellectual performance of the user may then be assessed with respect to the visual content that is being presented to the user during a writing assessment. Parameters indicating the intellectual performance of the user may include the amount of time taken by the user to respond to the visual content that is presented on the display 101 and the amount of time that the user takes to complete a task as instructed by the visual content presented on the display 101. The intellectual performance of the user may further indicate the decision-making performance of the user, for example the speed at which the user reacts in response to visual content presented on the display 101. One or more of these parameters may be recorded by the sensor 102 or 106. Further parameters indicating the intellectual performance of the user may be recorded in addition to these examples.

The memory 103 is configured to store a number of predetermined relationships linking the chirographic and/or intellectual performance of a user in response to known visual content that can be shown on the display 101 to a change in a neurodegenerative disease. These relationships may describe the progression of general neurodegeneration, or the progression of a specific disease that a patient may have. Alternatively, these relationships may describe the regression of neurodegeneration or of a specific disease. In one example of the invention the predetermined relationships may describe the progression of Alzheimer's disease. In alternative examples the predetermined relationships may describe the progression of Parkinson's or Huntington's disease, Amyotrophic Lateral Sclerosis (ALS), motor neurone diseases, or an alternative neurodegenerative disease. The predetermined relationships may be specific to the user; for example, concerning the user's previous responses to previously presented visual content, or could be based on predetermined data from the general public or from clinical trials.

The memory 103 is electrically connected to the processor 104 to enable the exchange of data in both directions between the memory 103 and the processor 104. Although the memory 103 is illustrated in FIG. 1 as being comprised within the device 105, the memory 103 may alternatively be a cloud-based memory that is accessed via a remote network and may be therefore be located remotely from the device 105. The memory 103 may be associated with an application that is configured to be run on the device 105. The processor 104 may perform its functions by executing program code that is stored in a non-transient form in memory 103.

The processor 104 is electrically connected to both the display 101 and the sensor 102 or 106. The processor 104 may be connected via either a physical electrical connection, or a wireless connection. In the example described above where the sensor 106 is located in a second device 107, it is preferable that the sensor 102 or 106 is connected to the display 101 by means of a wireless connection. The processor 104 is configured to command the display 101 to present the predetermined visual content to the user. The processor 104 is further configured to receive data from the sensor 102 or 106, and to receive data regarding the visual content on the display 101. The processor 104 is also electrically connected to the memory 103 and is configured to compare these data inputs to the predetermined relationships stored in the memory 103 to establish the progression of neurodegeneration of the user.

In one example of the invention, the visual content that is presented on the display 101 of the device 105 comprises an interactive assessment. The visual content may be pre-programmed into an application to be run on the device 105. The assessment may comprise presenting a number of instructions for the user to perform a writing task. The writing task may comprise replicating an illustrated symbol, such as a letter. Alternatively, the writing task may comprise a grammatical assessment, or alternatively may assess the vocabulary of the user. In this example, the sensor 102 or 106 may track the writing performance of a user in response to the instructions presented on the display 101 and in particular the chirographic performance of the user using the parameters described above. The content of the assessment that is being presented on the display 101 may also be recorded in the memory 103.

In an alternative example, the visual content that is shown on the display 101 may be content that is typical of the normal use of the device 105 or 107. An example of visual content that constitutes normal use of the device 105 or 107 within the context of the invention may include the graphic user interface (GUI) of a note taking application on the first device 105. However, it should be appreciated that this is merely an example of visual content that constitutes normal use of the device 105 or 107, and that alternative visual content that provokes a user to write using the device 105 or 107, 106 may be used. In this example, the chirographic and/or intellectual performance of the user is recorded using the sensor 102 or 106 as the user performs normal writing tasks in reaction to content presented on the device. The content that is being presented on the display 101 of the device 105 is also recorded in the memory 103. In this example, the system may be configured so that the sensor 102 or 106 is constantly recording data from the user whilst they are using the device 105 or 107 normally.

FIG. 2 illustrates an example of a method of using the system illustrated in FIG. 1 to monitor the progression of neurodegeneration in a patient, where visual content is actively presented to the user and the reaction of the user to the content is recorded by the sensor 102 or 106. At step 201 the processor 104 controls the display 101 to present the programmed visual content to the user. This step may be initiated after a user opens an application associated with the visual content, or alternatively when the user issues a specific command to the computer system to present the visual content. As described above, the visual content may comprise an interactive assessment. The visual content may instruct the user to perform a first action, for example as part of the interactive assessment that is comprised within the visual content. This action may comprise responding to performing a grammar or vocabulary test or replicating a text symbol that is presented on the display 101. The command may be provided in the form of a text sentence, or alternatively a symbol for a user to replicate.

The user's chirographic and/or intellectual performance when they respond to the visual content that is presented on the display 101 is recorded by the sensor 102 or 106, and at step 202 this data is received by the processor 104. At step 203 the processor 104 compares the received data indicating the performance of the user in response to the visual content with a number of prestored relationships between a predicted response to the visual content and the progression of a neurodegenerative disease that are stored in the memory 103. This indication may be a discrete absolute score of neurodegeneration; for example, a score of 1 could indicate that there is no impairment and a score of 10 could indicate a maximum score of neurodegeneration for an individual. Alternatively, the indication could be a relative score of impairment, and could compare the previous performance of the user to their current performance to determine whether the performance has changed.

FIG. 3 illustrates an alternative exemplary method of using the system illustrated in FIG. 1. In this example, there is no active visual content presented to the user. The user may not issue any form of command to initiate this method. Instead, the sensor 102 or 106 is constantly monitoring the user's chirographic and/or intellectual performance as they write using apparatus comprising the sensor 102 or 106.

The sensor 102 or 106 records data regarding the chirographic and/or intellectual performance, of the user, and at step 301 this data is received by the processor. This step might be performed at regular time intervals, after a certain amount of data has been recorded or may be triggered by the occurrence of a specific event, such as the input of a key word on the device 105. This step may alternatively be performed at other times or may be performed continuously. In one example, where the sensor 102 is comprised within a writing apparatus 107, data is recorded by the sensor at a plurality of distinct points in time. The sensor 102 or 106 may be configured to start recording data when an application that is configured to be run on the device 105 is open.

At step 302 the processor 104 compares the received data indicating the chirographic and/or intellectual performance of the user in response to the content that is being presented to the user on the display 101 with a number of prestored relationships between a predicted response to the visual content and the progression of a neurodegenerative disease that are stored in the memory 103. In one example, the processor is further configured to collect data from the sensor at a plurality of distinct points in time, and to use a plurality of those data values to determine a change in the chirographic performance of the user over time. That change in chirographic performance can then be compared to a number of predetermined relationships that are stored in the storage medium associating the change in chirographic performance of the user over time with an indication of the change in the neurodegenerative disease of the user. This comparison can therefore be used to establish a change in the neurodegenerative disease of the user. As with the method described in FIG. 2, this indication may be a discrete absolute score of neurodegeneration; for example, a score of 1 could indicate that there is no impairment and a score of 10 could indicate a maximum score of neurodegeneration for an individual. Alternatively, the indication could be a relative score, and could compare the previous performance of the user to their current performance to determine whether the performance has changed. The indication provides a method for establishing the progression of neurodegeneration of the user.

The applicant hereby discloses in isolation each individual feature described herein and any combination of two or more such features, to the extent that such features or combinations are capable of being carried out based on the present specification as a whole in the light of the common general knowledge of a person skilled in the art, irrespective of whether such features or combinations of features solve any problems disclosed herein, and without limitation to the scope of the claims. The applicant indicates that aspects of the present invention may consist of any such individual feature or combination of features. In view of the foregoing description it will be evident to a person skilled in the art that various modifications may be made within the scope of the invention.

1. A computer system for monitoring a change in a neurodegenerative disease in a user, the computer system comprising:
a writing apparatus comprising a sensor for recording data values indicating the chirographic performance of the user; and
a storage medium configured to store a plurality of predetermined relationships associating the change in chirographic performance of the user over time with an indication of the change in the neurodegenerative disease of a user;
a processor connected to the sensor, wherein the processor is configured to:
collect data values from the sensor at a plurality of distinct points in time indicating the chirographic performance of the user at each distinct point in time;
use a plurality of data values collected from the sensor at each distinct point in time to determine a change in the chirographic performance of the user over time; and
compare the change in the chirographic performance of the user over time with the plurality of predetermined relationships that are stored in the storage medium to establish the change in the neurodegenerative disease in the user.

2. A computer system as claimed in claim 1, wherein the neurodegenerative disease comprises at least one of Alzheimer's disease, Parkinson's disease, or Huntington's disease.

3. (canceled)

4. (canceled)

5. A computer system as claimed in claim 1, wherein the system further comprises a display for presenting visual content to a user.

6. (canceled)

7. (canceled)

8. A computer system as claimed in claim 1, wherein the data values indicating the chirographic performance of the user comprise values representing one or more of the following parameters:
(i) the roundness or sharpness of letters,
(ii) the spacing between letters,
(iii) the slope of letters,
(iv) repetition or irregularity of elements,
(v) the pressure applied during writing,
(vi) the average size of letters,
(vii) the thickness of letters, and
(viii) the speed of formation of letters.

9. A computer system as claimed in claim 1, wherein establishing the change in the neurodegenerative disease comprises establishing a discrete absolute score of neurodegeneration.

10. A computer system as claimed in claim 1, wherein establishing the change in the neurodegenerative disease comprises establishing a patient-specific relative score of neurodegeneration.

11. A computer system as claimed in claim 1, wherein the change in the neurodegenerative disease is a progression or a regression of the neurodegenerative disease.

12. (canceled)

13. (canceled)

14. A computer system as claimed in claim 1, wherein the writing apparatus is a digital pen.

15. (canceled)

16. (canceled)

17. A computer system for monitoring a change in a neurodegenerative disease in a user, the computer system comprising:
a display for presenting visual content to a user;
a sensor for recording data indicating:
(i) chirographic performance of the user; and
(ii) intellectual performance of the user;
a storage medium configured to store a plurality of predetermined relationships associating the chirographic and intellectual performance with an indication of the change in the neurodegenerative disease of a user;
a processor connected to both the display and the sensor, wherein the processor is configured to:
control the display to present visual content to a user of the device;
receive data from the sensor indicating the chirographic and intellectual performance of the user in response to the visual content;
compare the received data indicating the chirographic and intellectual performance of the user in response to the visual content with a plurality of prestored relationships between a predicted response to the visual content and the change in a neurodegenerative disease that are stored in the storage medium to establish the change in the neurodegenerative disease in the user.

18. A computer system as claimed in claim 17, wherein the neurodegenerative disease is one of Alzheimer's disease, Parkinson's disease, and Huntington's disease.

19. (canceled)

20. (canceled)

21. A computer system as claimed in claim 17, wherein the visual content comprises instructions for a user to perform one or more writing assessment tasks.

22. A computer system as claimed in claim 21, wherein the one or more writing assessment tasks comprise assessing at least one of the grammar or the vocabulary of the user.

23. (canceled)

24. A computer system as claimed in claim 21, wherein the one or more writing assessment tasks includes assessing the speed with which the user is capable of forming sentences or words.

25. A computer system as claimed in claim 17, wherein assessing the chirographic performance of the user comprises assessing one or more of:
(i) the roundness or sharpness of letters,
(ii) the spacing between letters,
(iii) the slope of letters,
(iv) repetition or irregularity of elements,
(v) the pressure applied during writing,
(vi) the average size of letters,
(vii) the thickness of letters, and
(viii) the speed of formation of letters.

26. A computer system as claimed in claim 17, wherein assessing the intellectual performance of the user comprises assessing one or more of:
(i) Speed of writing,
(ii) Accuracy of grammar,
(iii) Range of vocabulary.

27. A computer system as claimed in claim 17, wherein assessing the intellectual performance of the user comprises assessing one or more of the amount of time taken by the user to respond to the visual content that is presented on the display and the amount of time that the user takes to complete a task as instructed by the visual content presented on the display.

28. A computer system as claimed in claim 17, wherein establishing the change in the neurodegenerative disease comprises establishing a discrete absolute score of neurodegeneration.

29. A computer system as claimed in claim 17, wherein establishing the change in the neurodegenerative disease comprises establishing a patient-specific relative score of neurodegeneration.

30. A computer system as claimed in claim 17, wherein the change in neurodegenerative disease is a progression or a regression of the neurodegenerative disease.

31. (canceled)

32. A computer system as claimed in claim 17, wherein the processor and the storage medium are comprised within a first device.

33. (canceled)

34. (canceled)

35. (canceled)

36. (canceled)
